(19) United States
(12) Patent Application Publication
(10) Pub. No.: US 2026/0166220 A1
(43) Pub. Date: Jun. 18, 2026

(54) APPLICATOR DEVICE FOR INSULIN PATCH PUMP WITH IMPROVED SAFETY FEATURES

(71) Applicant: Tandem Diabetes Care, Inc., San Diego, CA (US)

(72) Inventor: Vincent Vaucher, St-Sulpice (CH)

(21) Appl. No.: 19/418,294

(22) Filed: Dec. 12, 2025

Related U.S. Application Data
(60) Provisional application No. 63/733,641, filed on Dec. 13, 2024.

(51) Int. Cl. A61M 5/158

(52) U.S. Cl. CPC A61M 5/158; A61M 2005/1585; A61M 2207/00

(57) ABSTRACT

Embodiments disclosed herein are directed to an applicator for inserting a cannula into a body of the user for delivering medication from a medication infusion device to the user. Features of the applicator device provide a more reliable, secure and safe insertion process.

### PRIORITY

The present application claims the benefit of U.S. Provisional Patent Application No. 63/733,641 filed Dec. 13, 2024, which is hereby incorporated herein by reference in its entirety.

### TECHNICAL FIELD

The present disclosure relates generally to ambulatory infusion pumps and, more particularly, to applicators for attaching ambulatory infusion pumps to user's body.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

For patch pumps, a transcutaneous cannula generally needs to be inserted into the skin for delivering medication transcutaneously from an external fluid reservoir associated with the patch pump that is in fluid communication with the cannula. Thus, the cannula needs to be inserted into the skin before delivering the medication. Patch pump systems may include an applicator that can simultaneously insert the cannula into the user's skin and affix an adhesive pad onto the user's skin that holds the patch pump.

### SUMMARY

Embodiments disclosed herein are directed to an applicator for inserting a cannula into a body of the user for delivering medication from a medication infusion device to the user. Features of the applicator device provide a more reliable, secure and safe insertion process.

According to one aspect, an applicator device includes a cannula and an insertion needle configured to insert the cannula into a body of a user. The applicator can include features that prevent the insertion needle from becoming loose within the applicator following actuation. This provides a safer process for the user because the insertion needle cannot become dislodged from the applicator, which would be an injury hazard for the user and/or others.

In an embodiment, an applicator for inserting a cannula into a body of a patient can include an applicator housing, a cannula assembly including a cannula and an insertion needle having a sharpened distal tip inserted into the cannula. An actuation assembly can be configured to actuate the insertion needle to extend the sharpened distal tip of the insertion needle from within the applicator housing to insert the cannula into a body of the user and to retract the sharpened distal tip back into the applicator housing. An internal component within the applicator housing can be configured to prevent the insertion needle from becoming disconnected from the actuation assembly within the applicator housing following insertion of the cannula.

In an embodiment, an applicator for inserting a cannula into a body of a patient can include a cannula and an insertion needle having a sharpened distal tip to insert the cannula into the body of the patient and a proximal loop. An actuation assembly can be configured to actuate the insertion needle to insert the cannula. The actuation assembly can include a link having a pin attached to the proximal loop to connect the link to the insertion needle. An internal component can prevent the proximal loop from being disconnected from the pin.

In embodiments, a method of manufacturing an applicator for insertion a cannula into a body of a patient can include orienting an insertion needle at an assembly angle relative to a horizontal position of a link disposed with an applicator housing with the insertion needle partially inserted through a cannula shaft of a cannula assembly. The insertion needle can then be slid towards the link to position a proximal loop of the insertion needle adjacent a pin extending from the link. The insertion needle and the cannula assembly can then be rotated from the assembly angle to an angle parallel with the horizonal position of the link such that the pin is inserted through the proximal loop. The cannula assembly can then be slid proximally towards the link such that the insertion needle slides distally through the cannula shaft.

In an embodiment, an applicator for inserting a cannula into a body of a patient can include an applicator housing, a cannula assembly including a cannula and an insertion needle having a sharpened distal tip inserted into the cannula. An actuation assembly can be configured to actuate the insertion needle to extend the sharpened distal tip of the insertion needle from within the applicator housing to insert the cannula into a body of the user and to retract the sharpened distal tip back into the applicator housing. An internal component within the applicator housing can be configured to maintain a connection between the insertion needle and the actuation assembly within the applicator housing following insertion of the cannula.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A and 3B are, respectively, a perspective view and an exploded view of the pad and applicator of FIG. 1.

FIGS. 4A-4B schematically depict a portion of an applicator of a medical infusion system.

FIG. 5 depicts an insertion needle for use with an applicator of a medical infusion system.

FIGS. 6A-6D schematically depict a subassembly of an applicator of a medical infusion system.

FIG. 7 schematically depicts a portion of an applicator of a medical infusion system incorporating the subassembly of FIGS. 6A-6D.

FIG. 8 depicts an exploded view of an applicator of a medical infusion system.

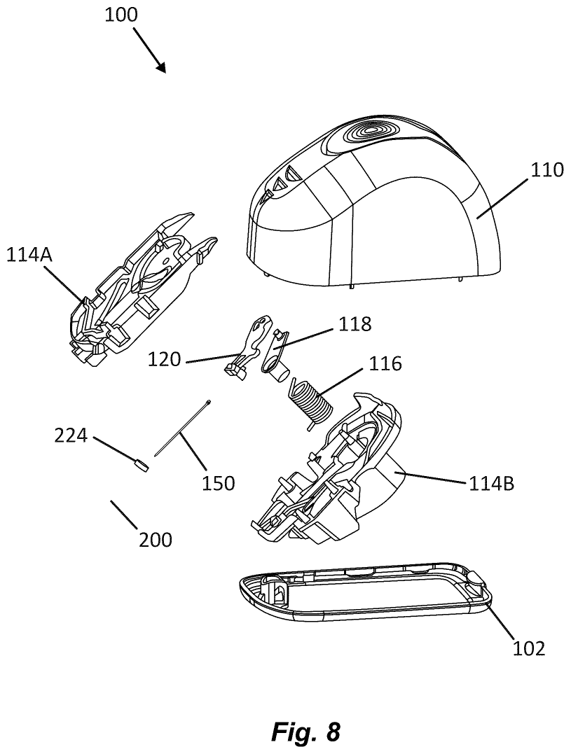

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

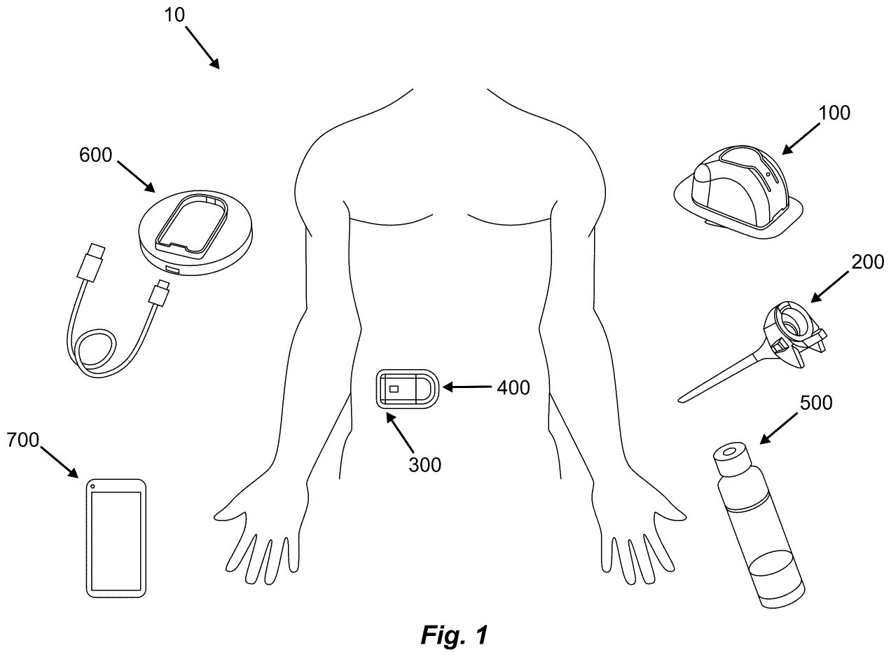

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula assembly 200, pump 300, cap 400, cartridge 500, charging system 600 and/or software application 700. Preferably, applicator 100, cannula assembly 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula assembly 200 and a pad to a wearer and to deliver medication through cannula via a patch pump coupled to the pad. Further details regarding such a system can be found in U.S. Patent Publication No. 2022/0379014, which is hereby incorporated herein by reference in its entirety.

Applicator 100 is configured to apply an adhesive pad to the wearer and, upon actuation, to insert cannula 200 into the wearer. The pad is configured to be secured to the wearer for a period of time, e.g., at least 3 days, 7-10 days, etc., and then may be replaced by a similar pad using a similar applicator. The pad may include a pad skeleton having one or more locking mechanisms that are configured to couple the pad to applicator 100 for insertion of cannula 200 or to the assembled pump for delivery of medication. Applicator 100 may include an internal component configured to support an insertion mechanism designed to insert cannula 200 through the skin of the wearer via rotational movement and to guide and orient cannula 200 during insertion.

Preferably, applicator 100 is designed to suppress noise during insertion. The insertion mechanism may include an applicator needle configured to pierce the wearer's skin and a biasing member, which may be coupled to one or more links configured to interact with cannula 200 and the applicator needle. Upon actuation by the wearer, the insertion mechanism preferably rotates and applies a distal force on cannula 200 and the applicator needle within cannula 200, such that cannula 200 is inserted through the wearer's skin. Cannula assembly 200 may include a proximal cannula head configured to couple to one or more locking mechanisms on the pad skeleton and, at the same time, uncouple applicator 100 from the pad skeleton. The insertion mechanism further may be configured to continue rotating to withdraw the applicator needle from cannula 200 and to store the applicator needle within the applicator after cannula 200 is inserted.

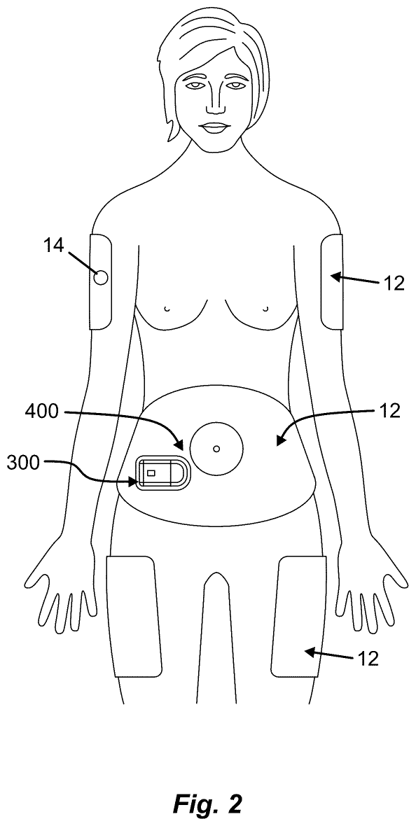

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

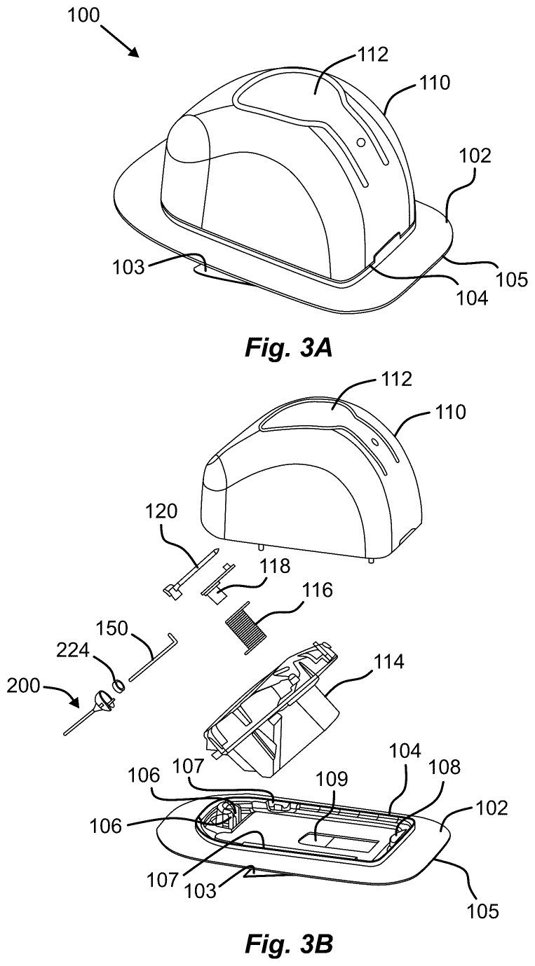

Referring now to FIGS. 3A and 3B, perspective and exploded views of an exemplary pad and applicator are described. Applicator 100 may transcutaneously apply a cannula, upon actuation by a user, which is designed to deliver doses of medication (e.g., insulin) from a patch pump configured to be removable coupled to the cannula. Advantageously, applicator further may apply a pad that is adhered to the wearer's skin and then coupled to the patch pump. For example, actuation of applicator 100 may both insert the cannula and cause the cannula to be locked to the adhesive pad in a single actuation. Further, applicator 100 may include internal components designed to minimize noise during the actuation process. For example, applicator 100 may avoid clicks and/or hard stops that make audible noises during insertion of the cannula.

In a pre-actuation state, applicator 100 may be coupled to pad 102 as shown in FIG. 3A. For example, applicator 100 may be coupled to pad 102 via pad skeleton 104 of pad, which is disposed on a first surface of pad 102. Skin-safe pad adhesive 105 may be disposed on a second, skin-facing surface of pad 102 such that the pump-pad assembly may be attached to a wearer for a period of time, for example, 3-5 days, 3-10 days, or 10 days or more. One or more release liners 103 may be attached to pad adhesive 105 until pad 102 is ready to be secured to the wearer. Pad skeleton 104 may be a frame with a shape designed to surround the pump-cap assembly so as to securely couple the adhesive pad to the pump-cap for wearing by the patient. Pad skeleton 104 may be designed to removably couple portions of pad 102 to applicator 100 in the pre-actuation state. For example, pad skeleton 104 may have one or more attachment mechanisms to lock pad 102 to applicator 100 and unlock upon actuation of applicator 100. Advantageously, the attachment mechanisms also may lock the cannula to pad 102 after actuation. As depicted in FIG. 3A, pad skeleton 104 may have pad attachments 106 at a first end of pad 102 and pad back clip 108 at a second end of pad 102. Pad attachments 106 and pad back clip 108 may interact with applicator 100 or a patch pump to lock the pad to applicator 100 or the patch pump. Pad attachments 106 may include at least two arms that protrude upwards from the pad and away from the skin surface of the wearer. Each arm may have an opening (e.g., slot) to receive extensions from the applicator during pre-actuation and extensions from the cannula post-actuation. Thus, the arms, which may have a U-shape, and openings may be used to lock to both the applicator and the cannula. Pad skeleton 104 may also include pad clips holes 107 disposed on the sides of pad skeleton 104. Pad clips holes 107 may be a hole or receptacle sized and shaped to interact with a corresponding feature of the pump-cap assembly such that the pump-cap assembly may be locked to the pad. Further, pad 102 may include pad opening 109 to allow direct sensing of the wearer's skin by one or more sensors of the pump. For example, the skin sensor(s) and/or the PPG sensor(s) may be positioned at pad opening 109 when the pump is coupled to the pad.

Applicator 100 may include applicator housing 110 and actuator 112. Applicator housing 110 is configured to house the mechanisms for inserting the cannula. After insertion of the cannula, internal component 114 is designed to withdraw and safely store the needle used to pierce the wearer's skin. Actuator 112, upon actuation, causes the cannula to be transcutaneously inserted into the wearer's skin. Actuation of actuator 112 also may unlock applicator 100 from pad 102. Actuation of actuator 112 also may lock the transcutaneously inserted cannula into pad 102. For example, actuation of applicator 100 may insert the cannula transcutaneously, unlock the applicator from the pad, and lock the cannula to the pad in a single actuation. Actuator 112 may release the internal mechanism disposed within applicator housing 110 when actuated by the wearer, thus causing the cannula to advance through the wearer's skin. Actuator 112 may be a button configured to be pressed by the wearer as illustrated, or may be a lever, snap, knob, or the like. The mechanism for inserting the cannula may include internal component 114, biasing member 116, and links 118 and 120, which are disposed within applicator housing 110, and are configured to advance cannula 200 through pad 102 and into the wearer's skin. The mechanism may further include applicator needle 150, which is configured to be disposed within cannula 200 during insertion and withdrawn from cannula 200 after insertion. Self-sealing septum 224 may be disposed within the cannula head of cannula assembly 200 in order to support and guide applicator needle 150 and minimize backflow out of cannula 200.

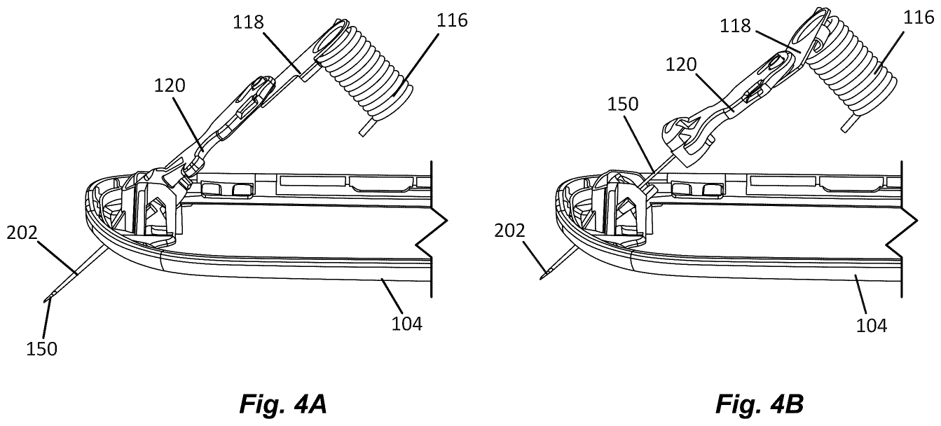

FIGS. 4A-4B schematically depict a portion of an applicator 100 for a medication infusion system illustrating features of a cannula insertion cycle 200. FIG. 4A depicts the insertion needle 150 at a maximum insertion depth with link 118 having rotated with respect to biasing member 116 and link 120 fully extended from link 118 to insert the cannula shaft 202 along with the needle 150 into the user's body. FIG. 4B depicts the applicator with the insertion needle 150 partially retracted and the cannula 202 remaining in the body. When the insertion needle 150 is fully extracted from the cannula assembly 200, the needle is in the fully retracted state.

As described above, a sharp insertion needle 150 is used to insert the cannula 200 into the body of the user. The needle 150 is then retracted back into the applicator 100. It has been found that during the cannula deployment process, the needle 150 can decouple from link 120 to which it is attached during this process. This can cause the needle 150 to immediately fall out of the applicator 100 or to be contained loosely and disconnected within the applicator 100 such that the needle 150 can fall out of an opening in applicator 100 at a later time. Either scenario could result in an injury to the user or an injury and/or spread of disease to another. It is therefore important that the needle 150 remain within the applicator 100 and not become capable of being accidentally dislodged from the applicator where it could cause injury.

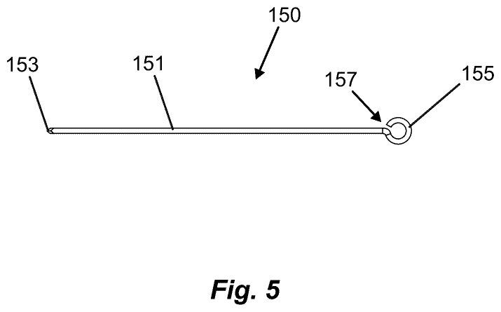

An example of an insertion needle 150 that can be used with embodiments disclosed herein that provide for enhanced needle safety is depicted in FIG. 5. Insertion needle 150 can include a shaft 151 having a sharpened distal tip 153. A proximal loop 155 can be disposed at an opposite end of shaft 151 from distal tip 153. In embodiments, proximal loop 155 can comprise a hooked, partially circular shape with an opening 157 adjacent shaft 151 or can be a fully closed loop.

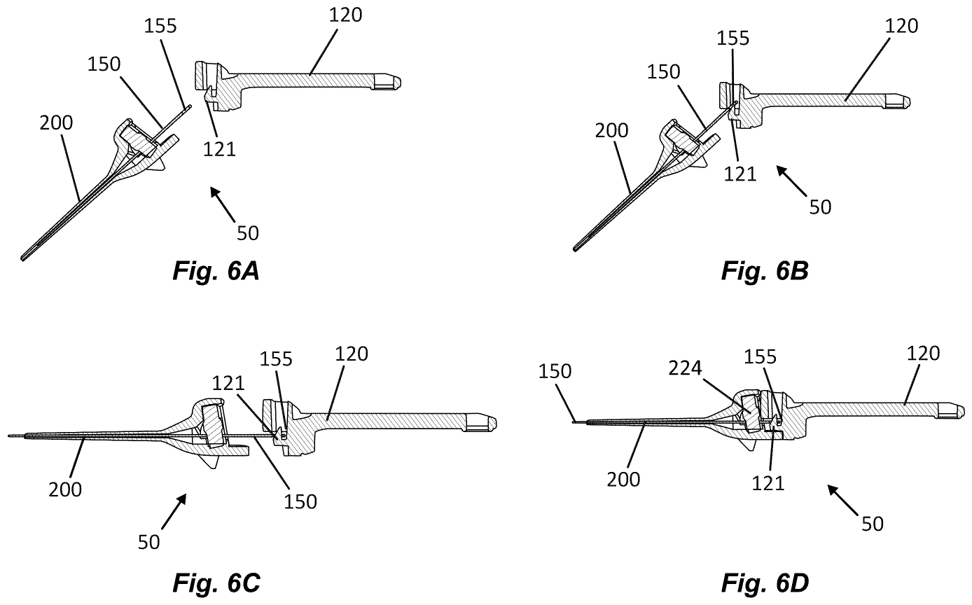

FIGS. 6A-6D schematically depict a needle assembly sequence that provides an insertion subassembly 50 that eliminates the possibility of the needle 150 exiting the applicator 100 after it is retracted. This sequence modifies how the needle 150 is assembled and oriented within the applicator 100 during manufacturing of the applicator 100 prior to actuation of the needle 150 to maintain a connected between needle 150 and link 120 such that the needle 150 cannot become disconnected from link 120 and loose within the applicator during or following actuation of the needle 150.

FIG. 6A depicts an initial orientation of the insertion needle 150 with the needle 150 partially inserted into the cannula 200. The insertion needle 150 is oriented at an assembly angle of generally about 30 degrees to 80 degrees relative to a horizontal position of link 120 and is spaced apart from link 120. In one embodiment, the assembly angle is 45 degrees. The insertion needle 150 is then slid back towards the link 120 until the proximal loop 155 of the insertion needle 150 is positioned adjacent a pin 121 or other projection on the link 120 as shown in FIG. 6B. The cannula 200 and needle 150 are then rotated to the horizonal position parallel with the link 120 as shown in FIG. 6C, which is the deployment angle between the needle 150 and the link 120 when the needle 150 is later deployed to insert the cannula 200. This rotation from the assembly angle to the deployment angle causes the proximal loop 155 on the needle 150 to be coupled with the pin 121 (i.e., the pin 121 is inserted through the loop 155). The cannula 200 is then slid back towards the link 120 as shown in FIG. 6D. The needle 150 will slide distally through the septum 224 and elongate shaft 202 of the cannula 200 as the cannula 200 is slid back with the needle 150 remaining in the same relative position to the link 120 and the proximal loop 155 remaining coupled to the pin 121. The is the final assembly step for this cannula 200, needle 150 and link 120 insertion subassembly 50. Friction between the septum 224 and needle 150 will enable the insertion subassembly 50 to remain in the assembled position shown in FIG. 6D during subsequent assembly steps of the applicator 100.

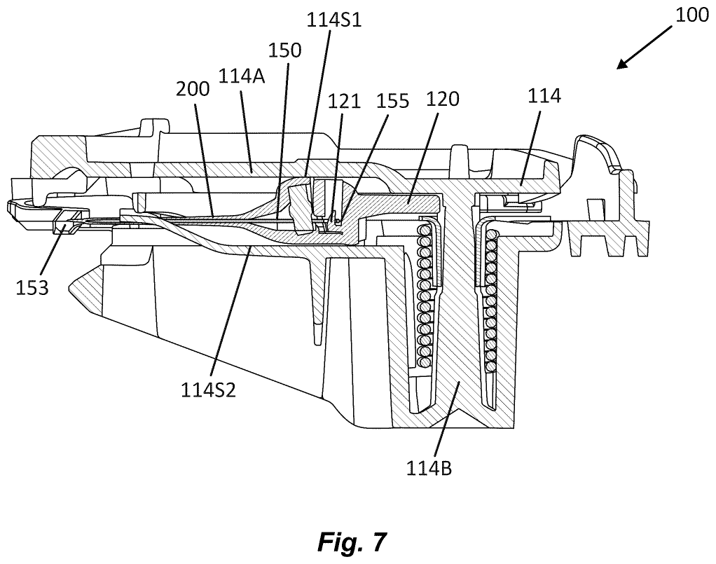

FIG. 7 is a portion of an assembled applicator 100 that depicts insertion subassembly 50 within applicator 100. When the applicator 100 is actuated to insert the needle 150 and cannula 200 into the user, the needle 150 is actuated in the direction in which the distal tip is oriented in FIG. 7 (i.e., horizontally to the left of the page). In this orientation, the proximal loop 155 on the needle 150 cannot detach from the pin 121 of link 120 because an upper surface 114S1 of internal component 114 (see also FIG. 3B) prevents the cannula 200, and therefore the needle 150, from moving upwardly to dislodge the proximal loop 155 from the pin 121. The subassembly 50 also is prevented from pivoting downwardly from the depicted deployment angle to the assembly angle depicted in FIGS. 6A-6C at which the proximal loop 155 may become disconnected from pin 121 by a bottom surface 114S2 of internal component that prevents such motion. As such, the insertion subassembly 50 is sandwiched between opposing surfaces 114S1, 114S2 of the portions 114A, 114B of the internal component 114 such that the proximal loop 155 cannot decouple from the pin 121, effectively creating a permanent connection between the needle 150 and the link 120 such that the needle 150 cannot become loose in the housing.

Applicator 100 disclosed herein therefore provides for enhanced safety by preventing the needle from coming loose within the applicator from which it could fall out and create an injury hazard.

In embodiments, an applicator for inserting a cannula into a body of a patient can include an applicator housing, a cannula assembly including a cannula and an insertion needle having a sharpened distal tip inserted into the cannula. An actuation assembly can be configured to actuate the insertion needle to extend the sharpened distal tip of the insertion needle from within the applicator housing to insert the cannula into a body of the user and to retract the sharpened distal tip back into the applicator housing. An internal component within the applicator housing can be configured to prevent the insertion needle from becoming disconnected from the actuation assembly within the applicator housing following insertion of the cannula.

In some embodiments, the internal component prevents the insertion needle from moving upwardly with respect to the actuation assembly.

In some embodiments, the internal component prevents the insertion needle from pivoting downwardly with respect to the actuation assembly.

In some embodiments, the internal component sandwiches the cannula assembly between opposing upper and lower surfaces.

In some embodiments, the internal component constrains the insertion needle and cannula assembly such that the insertion needle and cannula assembly can only move along an insertion angle within the applicator housing.

In some embodiments, the actuation assembly comprises a link and the insertion needle is connected to the link.

In some embodiments, the insertion needle comprises proximal loop inserted onto a pin of the link.

In some embodiments, the internal component prevents the insertion needle from becoming disconnected from the link.

In some embodiments, the internal component prevents the insertion needle from moving upwardly with respect to the link.

In some embodiments, the internal component prevents the insertion needle from pivoting downwardly with respect to the link.

In embodiments, an applicator for inserting a cannula into a body of a patient can include a cannula and an insertion needle having a sharpened distal tip to insert the cannula into the body of the patient and a proximal loop. An actuation assembly can be configured to actuate the insertion needle to insert the cannula, the actuation assembly including a link having a pin attached to the proximal loop to connect the link to the insertion needle. An internal component can be configured to prevent the proximal loop from being disconnected from the pin.

In some embodiments, the internal component prevents the proximal loop from being disconnected from the pin because a surface of the internal component prevents the cannula from moving upwardly with respect to the pin.

In some embodiments, the internal component prevents the proximal loop from being disconnected from the pin because a surface of the internal component prevents the cannula from being pivoted downwardly with respect to the pin.

In some embodiments, the internal component prevents the proximal loop from being disconnected from the pin by sandwiching the cannula between opposing surfaces of the internal component.

In some embodiments, the opposing surfaces prevent the cannula from moving upwardly with respect to the pin and from pivoting downwardly with respect to the pin.

In embodiments, a method of manufacturing an applicator for insertion a cannula into a body of a patient can include orienting an insertion needle at an assembly angle relative to a horizontal position of a link disposed with an applicator housing with the insertion needle partially inserted through a cannula shaft of a cannula assembly. The insertion needle can then be slid towards the link to position a proximal loop of the insertion needle adjacent a pin extending from the link. The insertion needle and the cannula assembly can then be rotated from the assembly angle to an angle parallel with the horizonal position of the link such that the pin is inserted through the proximal loop. The cannula assembly can then be slid proximally towards the link such that the insertion needle slides distally through the cannula shaft.

In some embodiments, the assembly angle is between about 30 degrees and 80 degrees.

In some embodiments, the assembly angle is about 45 degrees.

In some embodiments, the angle parallel with the horizontal position of the link is an angle at which the insertion needle is inserted into a user's skin upon actuation of the insertion needle to insert the cannula shaft into the user's skin.

In some embodiments, the cannula assembly is slid proximally towards the link until the insertion needle protrudes from a distal end of the cannula shaft.

In an embodiment, an applicator for inserting a cannula into a body of a patient can include an applicator housing, a cannula assembly including a cannula and an insertion needle having a sharpened distal tip inserted into the cannula. An actuation assembly can be configured to actuate the insertion needle to extend the sharpened distal tip of the insertion needle from within the applicator housing to insert the cannula into a body of the user and to retract the sharpened distal tip back into the applicator housing. An internal component within the applicator housing can be configured to maintain a connection between the insertion needle and the actuation assembly within the applicator housing following insertion of the cannula

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. An applicator for inserting a cannula into a body of a patient, comprising:
an applicator housing;
a cannula assembly including a cannula;
an insertion needle having a sharpened distal tip inserted into the cannula;
an actuation assembly configured to actuate the insertion needle to extend the sharpened distal tip of the insertion needle from within the applicator housing to insert the cannula into a body of the user and to retract the sharpened distal tip back into the applicator housing; and
an internal component within the applicator housing configured to prevent the insertion needle from becoming disconnected from the actuation assembly within the applicator housing following insertion of the cannula.

2. The applicator of claim 1, wherein the internal component prevents the insertion needle from moving upwardly with respect to the actuation assembly.

3. The applicator of claim 1, wherein the internal component prevents the insertion needle from pivoting downwardly with respect to the actuation assembly.

4. The applicator of claim 1, wherein the internal component sandwiches the cannula assembly between opposing upper and lower surfaces.

5. The applicator of claim 1, wherein the internal component constrains the insertion needle and cannula assembly such that the insertion needle and cannula assembly can only move along an insertion angle within the applicator housing.

6. The applicator of claim 1, wherein the actuation assembly comprises a link and wherein the insertion needle is connected to the link.

7. The applicator of claim 6, wherein the insertion needle comprises proximal loop inserted onto a pin of the link.

8. The applicator of claim 6, wherein the internal component prevents the insertion needle from becoming disconnected from the link.

9. The applicator of claim 8, wherein the internal component prevents the insertion needle from moving upwardly with respect to the link.

10. The applicator of claim 8, wherein the internal component prevents the insertion needle from pivoting downwardly with respect to the link.

11. An applicator for inserting a cannula into a body of a patient, comprising:
a cannula;
an insertion needle having a sharpened distal tip to insert the cannula into the body of the patient and a proximal loop;
an actuation assembly configured to actuate the insertion needle to insert the cannula, the actuation assembly including a link having a pin attached to the proximal loop to connect the link to the insertion needle; and
an internal component configured to prevent the proximal loop from being disconnected from the pin.

12. The applicator of claim 11, wherein the internal component prevents the proximal loop from being disconnected from the pin because a surface of the internal component prevents the cannula from moving upwardly with respect to the pin.

13. The applicator of claim 11, wherein the internal component prevents the proximal loop from being disconnected from the pin because a surface of the internal component prevents the cannula from being pivoted downwardly with respect to the pin.

14. The applicator of claim 11, wherein the internal component prevents the proximal loop from being disconnected from the pin by sandwiching the cannula between opposing surfaces of the internal component.

15. The applicator of claim 14, wherein the opposing surfaces prevent the cannula from moving upwardly with respect to the pin and from pivoting downwardly with respect to the pin.

16. A method of manufacturing an applicator for insertion a cannula into a body of a patient, comprising:
orienting an insertion needle at an assembly angle relative to a horizontal position of a link disposed with an applicator housing with the insertion needle partially inserted through a cannula shaft of a cannula assembly;
sliding the insertion needle towards the link to position a proximal loop of the insertion needle adjacent a pin extending from the link;
rotating the insertion needle and the cannula assembly from the assembly angle to an angle parallel with the horizonal position of the link such that the pin is inserted through the proximal loop; and
sliding the cannula assembly proximally towards the link such that the insertion needle slides distally through the cannula shaft.

17. The method of claim 16, wherein the assembly angle is between about 30 degrees and 80 degrees.

18. The method of claim 17, wherein the assembly angle is about 45 degrees.

19. The method of claim 16, wherein the angle parallel with the horizontal position of the link is an angle at which the insertion needle is inserted into a user's skin upon actuation of the insertion needle to insert the cannula shaft into the user's skin.

20. The method of claim 19, wherein the cannula assembly is slid proximally towards the link until the insertion needle protrudes from a distal end of the cannula shaft.
